(19) United States
(12) Patent Application Publication
(10) Pub. No.: US 2011/0281815 A1
(43) Pub. Date: Nov. 17, 2011

(54) Parental formulations of gemcitabine derivatives

(75) Inventors: Sayeh Ahrabi, Oslo (NO); Finn Myhren, Porsgrunn (NO); Ole Henrik Eriksen, Oslo (NO)

(73) Assignee: CLAVIS PHARMA AS, Porsgrunn (NO)

(21) Appl. No.: 13/121,660

(22) PCT Filed: Nov. 15, 2010
(86) PCT No.: PCT/NO10/00417

§ 371 Date: Aug. 8, 2011

Related U.S. Application Data
(60) Provisional application No. 61/262,999, filed on Nov. 20, 2009.

(51) Int. Cl. A61K 31/7068; A61P 35/00

(52) U.S. Cl. 514/49

(57) ABSTRACT

The present invention relates to parenteral formulations for certain long chain saturated and monounsaturated fatty acid derivatives of 2′,2′-difluorodeoxycytidine (Gemcitabine). In particular, the present invention relates to a parenteral pharmaceutical composition and a method of the preparation thereof, in order to accommodate therapeutically effective doses of the said derivatives ameliorating compliance in treatment of cancer.

### FIELD OF THE INVENTION

The present invention relates to a pharmaceutical composition comprising certain long chain saturated and monounsaturated fatty acid derivatives of 2′,2′-difluorodeoxy-cytidine (gemcitabine) as the active ingredient. In particular, the present invention relates to a pharmaceutical composition and the method of preparation thereof, suitable for parenteral administration of therapeutically effective doses of the said derivatives in order to ameliorate compliance in treatment of cancer.

### BACKGROUND OF THE INVENTION

Gemcitabine, which is a well known cytostatic compound, marketed under the trade name Gemzar by Eli Lilly & Co., has the formula:

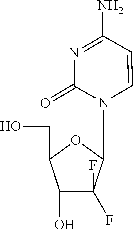

The active ingredients of the pharmaceutical composition of the present invention comprise gemcitabine derivatives of the formula I:

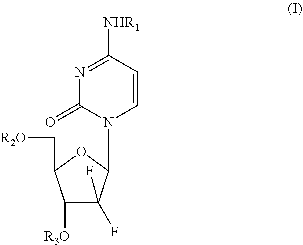

wherein R₁, R₂ and R₃ are independently selected from hydrogen and C₁₈- and C₂₀-saturated and monounsaturated acyl groups, with the proviso that R₁, R₂ and R₃ cannot all be hydrogen.

It is known from WO 98/32762 that the compounds of formula (I) are useful in treatment of cancer.

The cellular uptake of nucleosides and nucleoside analogues such as gemcitabine occurs mainly via the selective Nucleoside Transport (NT) receptor. Modulation/inhibition of this receptor may be seen as resistance to the drug in a clinical situation. This phenomenon can be observed in-vitro through addition of NT inhibitors. We have previously reported that our derivatives are not influenced by the presence of NT inhibitors, since the cytostatic activity of the preferred derivatives is conserved in the presence of such inhibitors (WO 98/32762).

There may also be other drug resistance mechanisms involved in cancer treatment. Multi drug resistance (MDR) is one of the principal reasons for failure of otherwise effective drugs. We have found that the derivatives of this invention seem not to be substrates for the MDR-pump, and hence circumvent this problem.

The half-life of gemcitabine in plasma is approximately 10 minutes, due to rapid deamination by the endogenous enzyme deoxycytidine deaminase to the corresponding uracil derivative (dFdU) (P. G. Johnston et aI, Cancer Chromatography and Biological Response Modifiers, Annual 16, 1996, Chap. 1, ed. Pinedo H. M. et al.).

In contrast, the derivatives of this invention are poor substrates for the deaminating enzyme. Consequently, the derivatives of this invention are more suited than gemcitabine itself for systemic or local treatment of malignant tumours.

However, formulation of a therapeutically effective amount of the poorly soluble derivatives of formula (I) into a pharmaceutical composition suitable for parenteral administration represents a problem. For the sake of intravenous administration of the said derivatives, the composition of the excipients should be selected so that the said derivatives were solubilised or formed nanosized particles. The gemcitabine derivatives of formula (I) are amphiphilic and have poor solubility both in water and in oils, which limits the choice of potential excipients that can solubilise them. If the formulation is a particulate system, there are certain requirements for the size of the particles in the formulations for intravenous administration. Moreover, parenteral products must be sterile and often sterile filtration is the only viable method for pharmaceutical particulate systems. This means that the particle size of these formulations must be smaller than 220 nm (0.22 μm), which is the pore size of the sterile filters. In practice and for an industrial scale process, the particles should be much smaller to avoid filter clogging.

Another issue is that since the said derivatives act as a prodrug for gemcitabine it is expected (and is recently confirmed, see example 14 in this document) that their clinical dose is in the same order of magnitude as that of gemcitabine. Gemzar is administered intravenously (i.v.) at a dose of 1000 mg/m² (3.3 mmol/m² of active), and the recommended dose for intravenous gemcitabine-5′-elaidic acid ester is 1250 mg/m² (2.4 mmol/m² of active). This means that for an average patient with a surface area of 1.8 m², the total dose of gemcitabine-5′-elaidic acid ester will be 2250 mg as a monotherapy.

This introduces even further challenges: a) requirement of increasing the concentration of the drug in the formulation in order to limit the parenteral administration of large volumes of liquids to the patients, and b) avoiding the use of additional functional excipients such as antioxidants and preservatives, which although added at small amounts, will add up to an unacceptable level of the total administered amount.

Finally, the derivatives of formula (I) are prone to hydrolytic degradation in physiological pH, the rate of which depends on the type of the derivative and the buffer. As an example, gemcitabine-5′-elaidic acid ester has a half life of approximately 30 minutes in pH 7.4. This represents further challenges both to the formulation and to the manufacturing process parameters. It is normally preferred that a pharmaceutical product be ready-to-use, but it is also possible to freeze dry the formulation in order to avoid degradation during the products shelf-life period. If ready-to-use, then the said derivatives should be protected from hydrolytic degradation in the aqueous environment of the parenteral formulation during its entire shelf-life period.

The present invention presents a solution to all the above problems.

### SUMMARY OF THE INVENTION

We have surprisingly found a pharmaceutical composition suitable for parenteral administration and a method of preparation for gemcitabine derivatives of formula (I) that results in ready-to-use high drug load aqueous nanoparticulate formulation based on phospholipids, with a drug to lipid molar ratio as high as 1:2, even more preferably as high as 1:1., where the said lipid nanoparticles protect the said derivative from hydrolytic degradation to gemcitabine for at least 38 months when stored at 2-8° C. under nitrogen blanket. Furthermore, the method uses natural phospholipids derived from egg yolk, does not incorporate any surfactant and results in micelle-like nanoparticles with volume based D_{(vol,0,99)} diameter of 5-20 nm, or intensity based Z-average based diameter of 30-50 nm as determined using instrumentation designed for small particle size analysis such as the Malvern Zetasizer Nano. Particles of this size can be easily sterile-filtered. Additionally, the method of preparation is an industrially scalable one, suitable for manufacture of aqueous sterile products.

Considering the described limitations of the formulations of U.S. Pat. No. 6,406,713 and U.S. Pat. No. 6,984,395 it was not expected that the pharmaceutical composition described in the present invention would result in the said unique characteristics. This is mainly attributed to the physicochemical characteristics of the preferred gemcitabine-5′-fatty acid ester and an improved method of manufacture compared to those described in the said publications.

### DETAILED DESCRIPTION OF THE INVENTION

It is a main objective of the present invention to provide a pharmaceutical composition based on natural phospholipids suitable for parenteral administration comprising gemcitabine derivatives of formula (I) as the active ingredient, which accommodates therapeutically effective doses of the said derivatives, being as efficacious as, or more efficacious than gemcitabine, in the treatment of cancer.

This and other objectives of the present invention are achieved by the pharmaceutical composition and method of preparation thereof as described in the attached claims.

1. Active Pharmaceutical Ingredient

According to an embodiment of the present invention a pharmaceutical composition, comprising a gemcitabine derivative of formula (I):

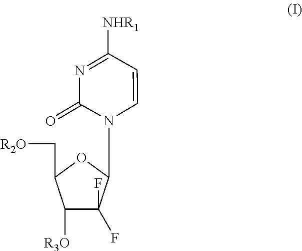

wherein R₁, R₂ and R₃ are independently selected from hydrogen and C₁₈- and C₂₀-saturated and monounsaturated acyl groups, with the proviso that R₁, R₂ and R₃ cannot all be hydrogen, or a pharmaceutically acceptable salt thereof as the active ingredient; wherein the active ingredient is dissolved or dispersed in phospholipids, is provided.

The active ingredient is prepared into a formulation comprising:
a) a solubilizer phospholipid selected from the group consisting of phosphatidylcholine, phosphatidylglycerol, phosphatidylethanolamine, phosphatidylinositol, phosphatidylserine, phosphatidic acid, lysophospholipids, sphingomyelin and cardiolipin in any form, including salted or desalted, hydrogenated or partially hydrogenated, natural, semisynthetic or synthetic;
b) a co-solubilizer selected from the group consisting of charged phospholipids;
c) an isotonicity agent and;
   wherein the active ingredient to phospholipid molar ratio is between 1:5 to 1:1 and the formulation has an average D₍ᵥₒₗ₎ particle size ranging between 2.5-30 nm.

In a preferred embodiment, the gemcitabine derivative of formula (I) has R₁ and R₃ as hydrogen and R₂ is a C₁₈- or C₂₀-saturated or monounsaturated acyl group.

Gemcitabine has three derivatisable functions, namely the 5′- and 3′-hydroxyl groups and the N4-amino group. Each group can selectively be transformed into an ester or amide derivative, but di-adducts (di-esters or ester-amides) and tri-adducts may be formed as well. In the case of the di- and tri-adducts the acyl substituent groups need not necessarily be the same.

Currently, the mono-acyl derivatives, i.e. with two of R₁, R₂ and R₃ being hydrogen, are preferred for use as the active ingredient of the present pharmaceutical composition. It is especially preferred that the monosubstitution with the acyl group should be in the 3′-O and 5′-O positions of the sugar moiety, with 5′-O substitution being most preferred.

The double bond of the mono-unsaturated acyl groups may be in either the cis or the trans configuration, although the therapeutic effect may differ depending on which configuration is used.

The position of the double bond in the monounsaturated acyl groups also seems to affect the activity. Currently, we prefer to use esters or amides having their unsaturation in the ω-9 position. In the ω-system of nomenclature, the position ω of the double bond of a monounsaturated fatty acid is counted from the terminal methyl group, so that, for example, eicosenoic acid (C20:1ω-9) has 20 carbon atoms in the chain and a single double bond is formed between carbon 9 and 10 counting from the methyl end of the chain. We prefer to use esters, ester-amides and amides derived from oleic acid (C18:1 ω-9, cis), elaidic acid (C18:1 ω-9, trans), eicosenoic acid(s) (C20:1 ω-9, cis) and (C20:1 ω-9, trans), and the amides and 5′-esters are currently the most preferred derivatives.

Esters, ester-amides and amides of gemcitabine derived from stearic acid (C18:0) and eicosanoic acid (C20:0) are advantageously used in some cases.

Gemcitabine (N4)-elaidic acid amide, gemcitabine-5′-elaidic acid ester and gemcitabine-3′-elaidic acid ester are among the most preferred derivatives and according to a preferred embodiment of the invention gemcitabine-5′-elaidic acid ester is the active ingredient of the pharmaceutical composition.

The derivatives of formula (I) are prepared according to methods known in the prior art (see WO 98/32762 for further details).

The pharmaceutical composition of the present invention is described herein as an aqueous formulation containing the active pharmaceutical ingredient (as described above), a solubilizer, a co-solubilizer and an isotonicity agent. According to a preferred embodiment of the invention the pharmaceutical composition comprises gemcitabine-5′-elaidic acid ester, phosphatidylcholine, phosphatidylglycerol, glycerol and water.

2. Solubilizer

According to a preferred embodiment of the present invention the phospholipids of the said pharmaceutical composition, comprise a neutrally charged phospholipid alone, or in combination with other phospholipids where at least one is a negatively charged phospholipid.

Phospholipids are natural components of cell membranes and are highly biocompatible. Phospholipids are amphiphilic molecules that spontaneously form bilayers in contact with water and upon further dilution turn into micro- and nanosized particles called liposomes. Lipophilic and amphiphilic molecules can be solubilised in the bilayers of phospholipids to a certain molar ratio without compromising the structure of the liposomes. The maximum drug concentration in such a formulation is dependent on the type and concentration of the phospholipids and the physicochemical characteristics of the active substance. The frequently used molar ratio of the drug to phospholipids in such formulations is in the range of 1:20.

Liposomes are prepared from natural or synthetic phospholipids, mainly phosphatidylcholine. For the purpose of stabilisation of the colloidal particles, a smaller amount of a negatively charged phospholipid such as phosphatidylglycerol may also be incorporated. The electrostatic repulsion due to the negative charge of the particles provides an effective barrier to aggregation and formation of larger particles.

Phospholipids can also form (mixed) micelles when combined with a surfactant such as bile salts, glycocholic acid, taurocholic acid, poloxamer, polysorbates, cremophore, sorbitan monolaurate, etc. In such case, the amphiphilic or lipophilic drug may be solubilised in the micelles in higher molar ratios than expected from liposomes. The reason is that due to the absence of the water core and the lack of organised structure of the liposomal bilayer, micelles permit a greater concentration of phospholipids, and hence the amphiphilic/lipophilic drug, per unit volume of their nanoparticle than liposomes do. Nevertheless, since surfactants are generally considered to exhibit concentration-dependent toxic adverse effects, the addition of these excipients to pharmaceutical formulations should be limited to a minimum.

The lipid nanostructures of the formulation may comprise, but are not restricted to, the following phospholipids, which function as solubilizers, bilayer-forming or micelle-forming excipients: phosphatidylcholine, phosphatidylglycerol, phosphatidylethanolamine, phosphatidylinositol, phosphatidylserine, phosphatidic acid, lysophospholipids, sphingomyelin, cardiolipin. The phospholipids may be in any form, including salted or desalted, hydrogenated or partially hydrogenated, natural, semisynthetic or synthetic. Also, attachment of hydrophilic polymers such as polyethyleneglycol (PEG) to the phospholipids in order to avoid rapid clearance by the reticuloendothelial system (RES) is possible.

In a preferred embodiment natural unsaturated phospholipids derived from hen egg are used alone or in combination as the solubilizer.

In still another embodiment of the invention the natural egg phospholipids comprise a zwitterionic phospholipid which is neutral in the pH range of 6-8 such as egg phosphatidylcholine. In a preferred embodiment, the solubilizer is purified hen egg phosphatidylcholine which is more than 96.0% phosphatidylcholine and not more than 1.0% lysophosphtidylcholine, not more than 1.0% sphingomyelin, and not more than 0.1% phosphatidylethanolamine

3. Co-Solubilizer

The formulations of the present invention contemplate use of a co-solubilizer. The co-solubilizer may be any suitable charged phospholipid. In a preferred embodiment, the phospholipid is negatively charged. In a more preferred embodiment the co-solubilizer phospholipid is negatively charged in the pH range of 6-8, such as hen egg phosphatidylglycerol which is more than 98.0% phosphatidylglycerol sodium salt, not more than 1.5% phosphatidic acid, not more than 0.5% lysophosphatidylglycerol, and not more than 0.5% phosphatidylcholine

4. Isotonicity Agent

In one embodiment of the invention, an isotonic agent is included in the pharmaceutical composition. An isotonic agent includes, but is not limited to, glycerol, propyleneglycol, polyethyleneglycol, poloxamers, polyols, carbohydrates, sugars, dextrans, aminoacids or proteins, organic or inorganic salts, and a mixture thereof. In a preferred embodiment, the isotonicity agent is glycerol.

5. Optional Excipients

In one embodiment, a sterol is added. In a preferred embodiment, this sterol is cholesterol.

In one embodiment, other components such as antioxidants, more preferably α-tocopherol, or fatty acids are added.

In another embodiment, a cryoprotectant is added in the pharmaceutical formulation to facilitate freeze drying. A cryoprotectant includes, but is not limited to, maltose, cellobiose, lactose, xylobiose, sucrose, trehalose, mannitol or dextran.

In yet another embodiment, the isotonic agent is also a cryoprotectant and is added in the pharmaceutical formulation both for isotonicity and to facilitate freeze drying. In a preferred embodiment, this isotonic and cryoprotectant agent is a disaccharide such as lactose, trehalose, sucrose, mannitol and the like.

6. Manufacture of the Formulation

In one embodiment of the invention, the composition of the excipients, the drug to lipid ratio and the method for manufacture is selected to favour a liposomal structure. In a particularly preferred embodiment, the said parameters are selected to favour micellar nanoparticles, or a combination of micelles and liposomes.

Some of the excipients of the pharmaceutical composition are selected to solubilise or to increase the solubility of the compounds of formula (I). In one embodiment the said excipient might be removed from the final product. In a preferred embodiment, this excipient is ethanol and is largely removed from the final product.

The pharmaceutical composition according to the invention is solid, semi-solid or liquid, preferably in liquid form, and may be presented in discrete units such as vials, infusion bags or the like. The pharmaceutical form of the final composition is a nanosized suspension or dispersion, either liposomes or micelle-like nanoparticles, or a combination of both.

Methods of preparing nonliposomal lipid complexes with high drug to lipid ratio has been described in U.S. Pat. No. 6,406,713 and U.S. Pat. No. 6,984,395. These so-called high drug:lipid complexes (HDLCs) are described in the first patent and are based on use of synthetic saturated phospholipids (combination of a phosphatidylcholine and a phosphatidylglycerol in a preferred molar ratio of 7:3). These carefully selected phospholipid complexes can accommodate up to 50 mole percent of a lipophilic drug without addition of any surfactant. Nevertheless, the size of HDLCs in this patent is reported to be in the micron range excluding the application of such a formulation for intravenous administration. A separation method for selecting the smaller fraction of the particles has been described, which introduce an additional time-consuming step to the method of manufacturing the product.

U.S. Pat. No. 6,984,395 further describes that by altering the processing conditions and the presence or absence of salts in the buffer one can choose between liposomal formulation or HDLCs with micellar structure. The inventors showed that these structures are nonliposomal (no bilayers were detected), and depending on the manufacturing process the size of HDLCs can be as small as 10-20 nm. The major draw-back of this latter micellar formulation compared to the larger particles described in the former patent is that the drug to lipid molar ratio is much lower, namely in the range of 1:7 to 1:10. The inventors explain that increasing the amount of lipids compared to the drug contributes to improvement of the sterile filterability of the product due to formation of smaller particles. Nonetheless, filtration volumes of only 500 mL were achieved with this approach. For routine commercial production of a drug product much larger volumes would be required.

The term “final pharmaceutical composition” as used herein refers to the prepared pharmaceutical composition that can be directly administered to the patient. This means that if the pharmaceutical composition is a freeze dried solid, the final pharmaceutical composition would refer to the reconstituted solution of the said formulation according to presettled instructions.

The term “therapeutically effective amount” as used herein refers to from about 0.001 to 10 grams per day of a gemcitabine derivative of formula (I) or a pharmaceutically acceptable salt thereof, more preferred from about 10 mg to 6 grams per day of a gemcitabine derivative of formula (I) or a pharmaceutically acceptable salt thereof, in a formulation containing 0.001-80% of the said derivative or salt thereof formulated for parenteral administration.

The amount of the phospholipid phase in the final pharmaceutical composition may vary from about 0.1% to 50%, preferably 1-15%, and more preferably 5-12%. In the most preferred but not limiting embodiment, the amount of the phospholipid phase is 9.5-10% of the final pharmaceutical composition. All subranges from 0.1% to 50% are included as part of the invention.

The molar ratio of the gemcitabine derivative of formula (I) to the total amount of the phospholipids in the final pharmaceutical composition may vary from 1:130 to 1:1, preferably 1:70 to 1:2. Another preferred range includes 1:6.6 (corresponding to 10 mg/ml gemcitabine derivative to lipid in final formulation) to 1:1.9 (corresponding to 35 mg/ml gemcitabine derivative to lipid in final formulation). Another preferred range includes 1:5.3 (corresponding to 12.5 mg/ml gemcitabine derivative to lipid in final formulation) to 1:2.2 (corresponding to 30 mg/ml gemcitabine derivative to lipid in final formulation). Another preferred range is 1:5 to 1:2. The most preferable range is 1:5 to 1:1. Another most preferred molar ratio of the gemcitabine derivative of formula (I) to the total amount of the phospholipids is 1:4.4 (corresponding to 15 mg/ml gemcitabine derivative to lipid in final formulation). All subranges between 1:130 and 1:1 are included as part of the invention.

The molar ratio of egg phosphatidylcholine to egg phosphatidylglycerol in the composition may vary from 1:1 to 99:1, preferably 2:1 to 80:1, with the most preferable ratio being 25:1. Another preferred range includes 15:1 to 40:1, more preferably 20:1 to 30:1, even more preferably 23:1 to 27:1. All subranges between 1:1 and 99:1 are included as a part of the invention.

Cholesterol may be added to the phospholipids in a molar ratio of 0.05:1 to 1:1, more preferably 0.2:1 to 0.5:1.

The amount of glycerol as the isotonic agent is adjusted to provide isoosmolar condition in the final pharmaceutical composition, and may or may not be added. If added, the amount may vary between approximately 50 mM to 350 mM depending on the other constituents of the formulation. In a preferred embodiment, the amount of glycerol is 260-300 mM, especially preferred amount is 285 mM. All subranges between 50 and 350 mM are included as a part of the invention.

The amount of the disaccharide used as isotonic/cryoprotective agent may vary between 1 to 50% of the final pharmaceutical composition, more preferably 5 to 15% and most preferably 7-10%. All subranges between 1 and 50% are included as part of the invention.

In another embodiment, the molar ratio of the isotonic/cryoprotective agent to total phospholipids is between 10:1 and 1:5, more preferably 5:1 to 1:1. All subranges between 10:1 and 1:5 are included as part of invention.

The present invention also provides a process for the preparation of a pharmaceutical composition as mentioned above. The said process comprises the steps of dissolving the phospholipids and the gemcitabine derivative of formula (I) in a suitable water-miscible organic solvent. Examples of water-miscible solvents are acetone, acetonitrile, dimethylformamide, ethylene glycol, glycerol, methanol, 1-propanol, 2-propanol, ethanol and DMSO. Phospholipids and the gemcitabine derivative of formula (I) may be dissolved in the same or in different water-miscible organic solvents as long as both organic solutions can be mixed together. Under carefully selected conditions, the said organic solution is then injected into an aqueous solution whereupon the lipid nanoparticles are formed. The size and structure of the nanoparticles in the said “intermediate bulk solution” are determined by the formulation and the injection parameters. One important parameter is the type and concentration of the organic solvent in the intermediate bulk solution. In one preferred embodiment, the organic solvent is ethanol used in an amount of 5 to 40%, more preferably 10 to 30% of the intermediate bulk solution. All intermediate values between 5 and 40% is covered by the present invention. Finally, the intermediate bulk solution is subjected to homogenization and removal of the organic solvent.

In a preferred procedure, the active pharmaceutical ingredient, solubilizer (preferably purified phosphatidylcholine) and co-solubilizer (preferably phosphatidylglycerol) are dissolved in ethanol and then injected into an aqueous solution containing water and the isotonicity agent (preferably glycerol) to form an intermediate bulk solution.

In one embodiment, the intermediate bulk solution is homogenised using conventional equipment until the aimed particle size is achieved, and then the bulk solution is concentrated to the final volume by tangential flow filtration and the organic solvent is removed by further diafiltration. In another embodiment, the said bulk solution is first concentrated to the final or an intermediate volume, thereafter homogenised using conventional techniques and equipment until the desired particle size is achieved. The bulk solution is thereafter, if necessary, concentrated further and the organic solution is finally removed by diafiltration. It is also possible to combine the final concentration and diafiltration in one step.

In a preferred procedure, the intermediate bulk solution is subjected to high pressure homogenization (typically between 625-1000 bar) for several passes until no further change is particle size is observed is subsequent passes. The particle size may be conveniently measured by laser light scattering to produce a volume-based diameter measurement −D₍ᵥₒₗ₎. The particles formed according to the manufacturing process above have a D₍ᵥₒₗ₎ (measured as the percentage of particles that have a size smaller than the indicated range) as follows:

D_{(vol,0,99)}: 5-11 nm

D_{(vol,0,90)}: 4-7 nm

D_{(vol,0,50)}: 2.5-5 nm

We estimate that the majority of the particles fall within a size range of D₍ᵥₒₗ₎ 2.5-30 nm.

Alternatively, the particle size as measured by the laser light scattering technique may also be expressed as an intensity based mean hydrodynamic volume (Z-average). One skilled in the art will appreciate the different absolute values reported using volume and intensity based units for particle size analysis. The particles formed according to the manufacturing process above have a measured Z-average range as follows:

Z-average: 20-50 nm

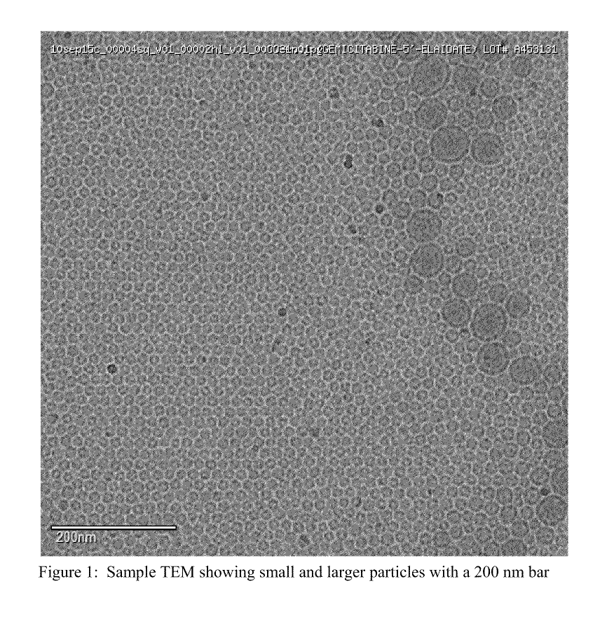

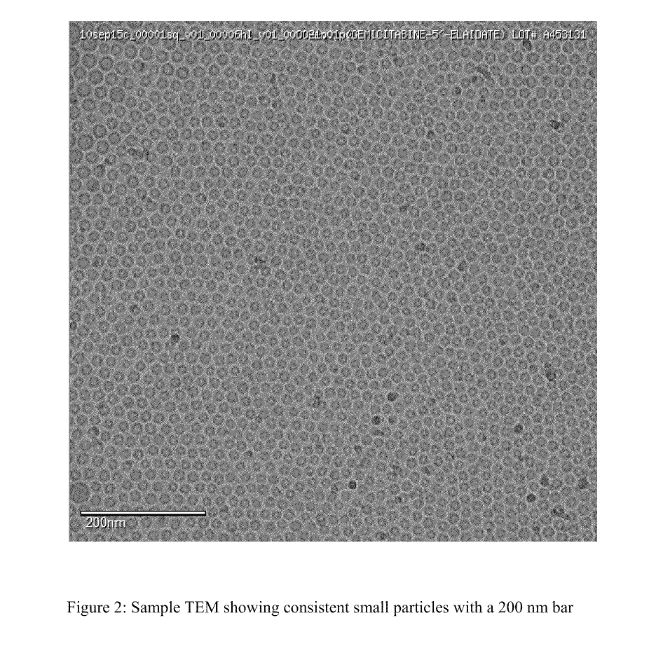

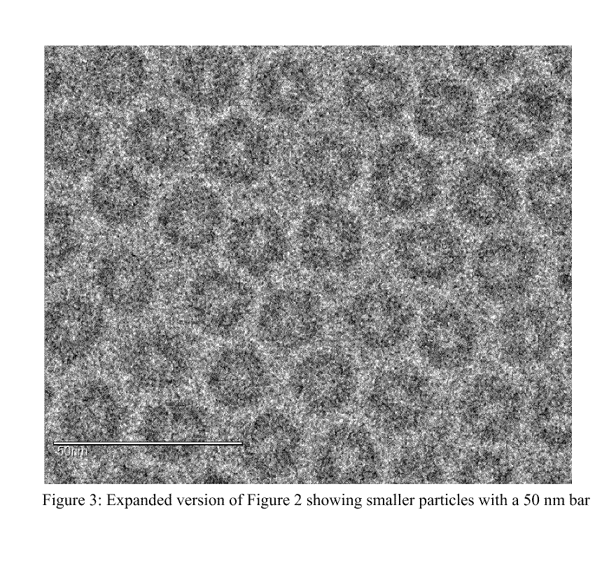

Physical measurement of particle size may also be performed, albeit on a much smaller evaluation sample. Transmission electron microscopy of particles held in vitreous ice have been evaluated. Particles appear to be primarily small with an observed diameter of between 15-20 nm with a small number of larger particles with observed diameters up to 50 nm. FIGS. 1 and 2 provide sample transmission electron micrographs (TEMs) of drug product prepared as described in Example 1. FIG. 3 shows an enlargement of the image from FIG. 2.

A pharmaceutical wetting agent may initially be added to the active substance before mixing with the lipid excipients. Examples of the wetting agents are polymers, surfactants, carbohydrates, polysaccharides, mineral solids, oils, alcohols or acids, organic or inorganic.

The nanoparticles of the final pharmaceutical composition are either liposome-like, meaning vesicles surrounded by phospholipid bilayer, or micelle-like, or a combination of both. The particle size distribution may be monomodal, bimodal or even multimodal, provided this does not impact the sterile filterability of the bulk solution. The particle size of the final pharmaceutical composition as determined by laser light scattering and expressed in volume based diameter units may be in a range of 2.5 nm to 220 nm. In one embodiment, the particle size distribution is monomodal and the average size ranges between 2.5 nm to 30 nm, or between 30 nm and 220 nm. In another embodiment, the particle size distribution is bimodal and has 2 distributions, one 2.5 nm-30 nm and the second 30-220 nm. In yet another embodiment, the particle size distribution is multimodal and the distributions have averages between 2.5 nm and 220 nm. In one preferred but not limiting embodiment, the particle size distribution of the final pharmaceutical composition is bimodal, where the main particle fraction has an average size of 2.5-25 nm, and the second minor particle fraction has an average particle size of 40-120 nm. All intermediate particle size values between 2.5 and 220 nm are covered by this invention. Alternative measurement techniques or expression units may result in small changes in the absolute ranges described above, however the measured particle sizes are consistently less than 200 nm in diameter.

7. Dosing

The pharmaceutical compositions of this invention are useful in treating a wide variety of cancers and specifically including metastatic pancreatic cancer, non-metastatic pancreatic cancer, metastatic breast cancer, non-metastatic breast cancer, non-small cell lung cancer, uterine cancer, ovarian cancer, cervical cancer, prostate cancer, biliary tract cancer, head and neck cancer, lymphomas, myelomas, and soft tissue sarcomas. The pharmaceutical composition may also be used as monotherapy or in combination with other approved or experimental cancer therapies.

The present invention provides a pharmaceutical composition defined as aforesaid for use in treatment of cancer, and in particular treatment of the cancers indicated above.

The preferred dosing schedule for intravenous administration is 1250 mg/m² once weekly for daily for 3 weeks out of 4 weeks. Alternative dosing schedules may be appropriate for specific cancer types, or when gemcitabine-5′-elaidate is used in conjunction with other therapeutic agents.

In the following the invention will be further explained by examples. The examples are only meant to be illustrative and shall not be considered as limiting.

### EXAMPLES

### Example 1

Gemcitabine-5′-elaidic acid ester, egg phosphatidylcholine (EPC) and egg phosphatidylglycerol (EPG) at the molar ratios of 5.8:25:1, resulting in an active agent to lipid ratio of 1:4.4, were added to ethanol in a weight ratio of 1:7.2. The mixture was stirred under heating up to 50° C. until all solid material was dissolved.

The ethanol solution was thereafter injected into the glycerol/water (2.6% w/w) solution at 250 ml/min under stirring. The weight ratio of ethanol solution to the glycerol solution was 1:7.6. The bulk solution was concentrated by tangential flow filtration, and the concentrated bulk was processed 6 times through a homogeniser at 15-20° C. The resulting product was further concentrated by tangential flow filtration to the final batch volume of 20 L and final gemcitabine-5′-elaidate concentration of 15 mg/mL. The residual ethanol was then removed through a washing step by diafiltration, the final product was sterile filtered and aseptically filled in sterile vials, purged with nitrogen and sealed. The vials were stored at 2-8° C. protected from light, and the stability of the batch was monitored up to 38 months. During the course of this stability study, no changes in the content of gemcitabine-5′-elaidic acid ester was observed. The amount of the main degradation product, gemcitabine, was 0.03% after 38 months. The batch showed a bimodal particle size distribution; after 38 months the main fraction (99.7%) had a mean size of 4.3 nm, and the other fraction (0.3%) a mean size of 69 nm. At the time of manufacturing the D_{(vol,0,99)} for this product at was measured to be 11 nm, and the Z-average was measured to be 47 nm.

### Example 2

Gemcitabine-5′-elaidic acid ester, egg phosphatidylcholine (EPC) and egg phosphatidylglycerol (EPG) at the molar ratios of 13.5:25:1, resulting in an active agent to lipid ratio of 1:1.9, were added to ethanol in a weight ratio of 1:6. The mixture was stirred until all solid material was dissolved.

The ethanol solution was thereafter injected into the glycerol/water (2.6% w/w) solution under stirring. The weight ratio of ethanol solution to the glycerol solution was 1:8.7. The bulk solution was homogenized 2 times and then concentrated by tangential flow filtration. The concentrated bulk was then processed 4 times through a homogenizer. The resulting product was further concentrated by tangential flow filtration to the final batch volume and gemcitabine-5′-elaidate concentration of 35 mg/mL. The residual ethanol was then removed through a washing step by diafiltration, the final product was sterile filtered and filled in vials, purged with nitrogen and sealed. The measured D_{(vol,0,99)} for the batch was 7.2 nm. The measured Z-average intensity based particle size was 46 nm.

### Example 3

Gemcitabine-5′-elaidic acid ester, egg phosphatidylcholine (EPC) and egg phosphatidylglycerol (EPG) at the molar ratios of 13.5:25:1, resulting in an active agent to lipid ratio of 1:1.9, were added to ethanol in a weight ratio of 1:0.7. The mixture was stirred until all solid material was dissolved.

The ethanol solution was thereafter injected into the glycerol/water (2.6% w/w) solution immediately upstream of a homogenizer. The weight ratio of ethanol solution to the glycerol solution was 1:5.3. The homogenizer was utilized to both mix and reduce particle size under these operational conditions. The bulk solution was concentrated by tangential flow filtration, and the concentrated bulk was processed 12 times through a homogeniser. The resulting product was further concentrated by tangential flow filtration to the final batch volume and gemcitabine-5′-elaidate concentration of 35 mg/mL. The residual ethanol was then removed through a washing step by diafiltration and the final product was sterile filtered. The batch showed a bimodal volume particle size distribution; the main fraction (99.9%) had a size of 3.9 nm, and the other fraction (0.1%) a mean size of 79 nm. The measured Z-average intensity based particle size was 61 nm.

### Example 4

Gemcitabine-5′-elaidic acid ester, egg phosphatidylcholine (EPC) and egg phosphatidylglycerol (EPG) at the molar ratios of 13.5:25:1, resulting in an active agent to lipid ratio of 1:1.9, were added to ethanol in a weight ratio of 1:2.5. The mixture was stirred until all solid material was dissolved.

The ethanol solution was thereafter injected into the glycerol/water (2.6% w/w) solution immediately upstream of a homogenizer. The weight ratio of ethanol solution to the glycerol solution was 1:2.2. The homogenizer was utilized to both mix and reduce particle size under these operational conditions. The bulk solution was processed 3 times through a homogeniser. The resulting product was concentrated by tangential flow filtration to the final batch volume and gemcitabine-5′-elaidate concentration of 35 mg/mL. The bulk solution was then processed an additional 6 times through a homogenizer. The residual ethanol was then removed through a washing step by diafiltration and the final product was sterile filtered. The batch showed a bimodal volume particle size distribution; the main fraction (99.9%) had a size of 2.9 nm, and the other fraction (0.1%) a mean size of 42 nm. The measured Z-average intensity based particle size was 15 nm.

### Example 5

Gemcitabine-5′-elaidic acid ester, egg phosphatidylcholine (EPC) and egg phosphatidylglycerol (EPG) at the molar ratios of 13.5:25:1, resulting in an active agent to lipid ratio of 1:1.9, were added to ethanol in a weight ratio of 1:1.5. The mixture was stirred until all solid material was dissolved.

The ethanol solution was thereafter injected into the glycerol/water (2.6% w/w) solution immediately upstream of a homogenizer. The weight ratio of ethanol solution to the glycerol solution was 1:3. The homogenizer was utilized to both mix and reduce particle size under these operational conditions. The bulk solution was processed 3 times through a homogeniser. The resulting product was concentrated by tangential flow filtration to the final batch volume and gemcitabine-5′-elaidate concentration of 35 mg/mL. The bulk solution was then processed an additional 6 times through a homogenizer. The residual ethanol was then removed through a washing step by diafiltration and the final product was sterile filtered. The batch showed a bimodal volume particle size distribution; the main fraction (99.9%) had a size of 3.9 nm, and the other fraction (0.1%) a mean size of 44 nm. The measured Z-average intensity based particle size was 14 nm.

### Example 6

Gemcitabine-5′-elaidic acid ester, egg phosphatidylcholine (EPC) and egg phosphatidylglycerol (EPG) at the molar ratios of 15.4:25:1, resulting in an active agent to lipid ratio of 1:1.7, were added to ethanol in a weight ratio of 1:5.2. The mixture was stirred until all solid material was dissolved.

The ethanol solution was thereafter injected into the glycerol/water (2.6% w/w) solution immediately upstream of a homogenizer. The weight ratio of ethanol solution to the glycerol solution was 1:8.5. The homogenizer was utilized to both mix and reduce particle size under these operational conditions. The bulk solution was passed 2 times through a homogenizer and then concentrated by tangential flow filtration to a gemcitabine-5′-elaidate concentration of 40 mg/mL and residual ethanol removed via diafiltration. The bulk solution was then processed an additional 4 times through a homogenizer and sterile filtered and filled in vials, purged with nitrogen and sealed The measured D_{(vol,0,99)} for the batch was 9.1 nm. The measured Z-average intensity based particle size was 33 nm.

### Example 7

Gemcitabine-5′-elaidic acid ester, egg phosphatidylcholine (EPC) and egg phosphatidylglycerol (EPG) at the molar ratios of 23.2:25:1, resulting in an active agent to lipid ratio of 1:1.1, were added to ethanol in a weight ratio of 1:5.2. The mixture was stirred until all solid material was dissolved.

The ethanol solution was thereafter injected into the glycerol/water (2.6% w/w) solution immediately upstream of a homogenizer. The weight ratio of ethanol solution to the glycerol solution was 1:8.5. The homogenizer was utilized to both mix and reduce particle size under these operational conditions. The bulk solution was passed 6 times through a homogenizer and then concentrated by tangential flow filtration to a gemcitabine-5′-elaidate concentration of 60 mg/mL and residual ethanol removed via diafiltration. The bulk solution was then sterile filtered and filled in vials, purged with nitrogen and sealed. The measured Z-average intensity based particle size was 44 nm.

### Example 8

Thermal analysis by Differential Scanning calorimetry (DSC) of the formulation described in Example 1 was performed to confirm the storage and shipment temperature of the product. It was shown that the freezing point was low at −22° C., probably due to supercooling of water. The melting point was at approximately −3° C. This suggested that a storage and shipment temperature of 2-8° C. would not cause melting or freezing of the phospholipids and hence would not pose any negative impact on the structure of the particles.

Stability studies have been conducted on drug product batches manufactured using the procedure described in Example 1 under storage conditions of 2-8° C./ambient RH for up to 38 months, and under storage conditions of 25° C./60% RH for up to 9 months. The results from those studies demonstrate product consistency under the stated storage conditions for the evaluated intervals.

Stability studies have been conducted on drug product batches manufactured using the procedure described in Example 2 and 6 under storage conditions of 2-8° C./ambient RH for up to 6 months, and under storage conditions of 25° C./60% RH for up to 6 months. The results from those studies demonstrate product consistency with respect to gemcitabine-5′-elaidate content and particle size under the stated storage conditions for the evaluated intervals. No precipitation was observed under the described storage conditions.

### Example 9

Gemcitabine-5′-elaidic acid ester, egg phosphatidylcholine (EPC) and egg phosphatidylglycerol (EPG) at the molar ratios of 5.9:25:1, resulting in an active agent to lipid ratio of 1:1.4.5, were added to ethanol in a weight ratio of 1:14. The mixture was stirred until all solid material was dissolved. This solution was injected into acetate/sucrose buffer pH 5.0 using a controlled pore size injection device and the suspension was concentrated to the final volume to give a 15 mg/ml final concentration of gemcitabine-5′-elaidic acid ester. Finally the buffer was changed to glycerol/water 2.6% w/w. The particle size of this batch was 92 nm (Z-average) and polydispersity index was 0.24.

Another batch was manufactured by reducing the starting concentrations of the lipids and the drug by 20%, while keeping the rest of the parameters constant. The particle size of this batch was 53 nm (Z-average) and the polydispersity index 0.27.

It is assumed that the particles in both batches had liposomal structure. Both batches were stored at 2-8° C. and analysed 5 months later. The particle size remained unchanged in case of the 92 nm batch, while the batch with smaller particle size showed a bimodal distribution (indication of aggregation of the particles). In addition, both batches showed 4-5% degradation of gemcitabine-5′-elaidic acid ester after 5 months. Compared to the clinical batches explained in example 1, this formulation is much more instable.

### Example 10

Particle formation without the use of phospholipids was not successful. Gemcitabine-5′-elaidic acid ester was dissolved in ethanol in a weight ratio of 1:24. The mixture was stirred until all solid material was dissolved.

The ethanol solution was thereafter injected into a water for injection solution with vigorous mixing. The weight ratio of ethanol solution to the glycerol solution was 1:12. The mixture appeared to contain conglomerates as determined by visual inspection. Attempts at homogenization were unsuccessful indicating that gemcitabine-5′-elaidate was not capable of forming stable particles in the absence of phospholipids.

### Example 11

Mixed micellar formulations with phospholipids and non-ionic surfactants were not successful in preparation of high drug load formulations. Mixed micelles were prepared using a 1:1 weight ratio of egg lecithin:Tween 20 for a final concentration of 50 mg/mL dissolved solids. Attempts to load the mixed micelles with gemcitabine-5′-elaidate resulted in maximal concentrations of less than 5 mg/mL. Substitution of the surfactant component with glycholic acid resulted in maximal gemcitabine-5′-elaidate concentrations of 2 mg/mL.

### Example 12

Liposomal formulations with egg lecithin and oleic acid were not successful in preparation of high drug load formulations. Liposomes were prepared by means of dissolution of gemcitabine-5′-elaidate, egg lecithin, and oleic acid in a molar ratio of 1:14.4:2 in ethanol. The solvent was evaporated and the residual solids dispersed into 2.6% glycerol in water using an Ultraturrax followed by high pressure homogenization. Stable formulations could be achieved up to 3 mg/mL gemcitabine-5′-elaidate. Formulations with higher concentrations of gemcitabine-5′-elaidate were found to contain undissolved solids.

### Example 13

It was investigated if the unexpectedly high concentration (15 mg/mL-60 mg/mL) of the active compound achieved in the formulations described in Examples 1 through 7, and under section 6, Manufacture of the Formulation, was unique to a chemical compound as shown in Formula (I) or if the method was of a more general nature. The structurally similar compound elacytarabine (ara-C-5′-elaidic acid ester) as shown in Formula (II) was used as a related model substance. This model substance contains the same 5′-elaidic acid ester moiety as found in Formula (I) (R1=R3=H, R2=elaidate), and the same cytosine nucleoside ring system. The minor difference between the compounds described in Formula (I) and Formula (II) is the substitution at the 2′ position of the ribose ring for Formula (I) contains geminal fluorines, while Formula (II) contains H and OH.

Formula II

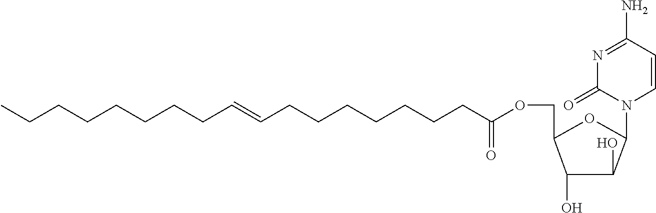

| | Elacytarabine concentration | | | |
|---|---|---|---|---|
| Lipids | 30 mg/mL | 20 mg/mL | 15 mg/mL | 10 mg/mL |
| Egg PC/egg PG | Precipitation | Precipitation during | Final content | Final content 8 mg/ml. |
| | during solvent | homogenisation | 12 mg/ml | Large lipid agglomerates |
| | injection | | Precipitation | seen after filtration, |
| | | | observed upon | Particle size |
| | | | storage at 2-8° C. | (Z-.avg) 97 nm |

Using consistent phospholipids and manufacturing techniques, formulations in which Formula II compound was included in excess of 20 mg/mL resulted in precipitation during processing. At target concentrations of Formula II compound of less than 15 mg/mL it was possible to prepare formulations, however these same formulations were found to be unstable during storage with respect to precipitation and agglomeration. It is concluded that the Formula I compound provides unexpectedly unique attributes of high loading and stability to the described lipid based formulations.

### Example 1

The intravenous formulation of gemcitabine-5′-elaidic acid ester as described in Example 1 was used in a phase I, first in human clinical study. The aims of this study were to determine the safety, toxicity, MTD (Maximum Tolerated Dose) and the RD (Recommended Dose) of gemcitabine-5′-elaidic acid ester, to describe its pharmacokinetic (PK) characteristics, and to assess its preliminary antitumor activity.

Gemcitabine-5′-elaidic acid ester was administered on days (d) 1, 8 and 15 every 4 week by a 30 min IV infusion. The dose range was from 30 to 1600 mg/m2/d. 43 patients were enrolled and the RD was established at 1250 mg/m²/d. The drug was well tolerated and the most frequent toxicities included nausea, fatigue, vomiting and anorexia, mostly of mild intensity. Stabilisation of disease (>3 months) reported in 7 patients (pancreas, colon and ovarian cancer) lasting between 3.5 to >8 months. One patient with ovarian cancer had a 28.3% reduction in tumor mass.

Gemcitabine-5′-elaidic acid ester was detected in plasma up to 24 hrs post-dosing. AUC for gemcitabine (dFdC) exposure was significantly higher than reported when gemcitabine was administered intravenousely at comparable dose levels. Urinary excretion of the main metabolite, dFdU, during the first 24 hrs was 48-71% of the dose.

1. A pharmaceutical composition comprising a gemcitabine derivative of formula I: 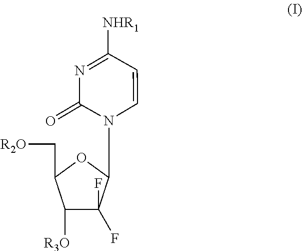
wherein R₁ and R₃ are hydrogen and R₂ is a C₁₈- or C₂₀-saturated and monounsaturated acyl group, or a pharmaceutical acceptable salt thereof as the active ingredient;
wherein the active ingredient is prepared into a formulation comprising:
a) a solubilizer phospholipid selected from the group consisting of phosphatidylcholine, phosphatidylglycerol, phosphatidylethanolamine, phosphatidylinositol, phosphatidylserine, phosphatidic acid, lysophospholipids, sphingomyelin and cardiolipin in any form, including salted or desalted, hydrogenated or partially hydrogenated, natural, semisynthetic or synthetic;
b) a co-solubilizer selected from the group consisting of charged phospholipids; and
c) an isotonicity agent;
wherein the active ingredient to phospholipid molar ratio is between 1:6.6 to 1:1 and the formulation has an average D₍ᵥₒₗ₎ particle size ranging between 2.5-30 nm.

2. The pharmaceutical composition of claim 1 wherein the active ingredient is gemcitabine-5′-elaidic acid ester.

3. The pharmaceutical composition of claim 1, wherein the solubilizer phospholipid comprises natural phospholipids derived from hen egg comprising a zwitterionic phospholipid which is neutral in the pH range of 6-8.

4. The pharmaceutical composition of claim 1, wherein the solubilizer phospholipid comprises purified phosphatidylcholine derived from hen egg.

5. The pharmaceutical composition of claim 1, wherein the co-solubilizer is hen egg phosphatidylglycerol.

6. The pharmaceutical composition according to claim 1, wherein the isotonic agent is glycerol.

7. The pharmaceutical composition according to claim 1, wherein the active ingredient is gemcitabine-5′-elaidic acid ester, the solubilizer is phosphatidylcholine, the co-solubilizer is phosphatidylglycerol, and the isotonicity agent is glycerol.

8. The pharmaceutical composition according to claim 1, wherein the active ingredient to phospholipid molar ratio is between 1:6.6 to 1:2.

9. A pharmaceutical composition comprising a gemcitabine derivative of
formula I: 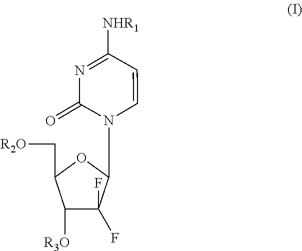
wherein R₁ and R₃ are hydrogen and R₂ is a C₁₈- or C₂₀-saturated and monounsaturated acyl group, or a pharmaceutical acceptable salt thereof as the active ingredient;
wherein the active ingredient is prepared into a formulation comprising:
a) a solubilizer phospholipid selected from the group consisting of phosphatidylcholine, phosphatidylglycerol, phosphatidylethanolamine, phosphatidylinositol, phosphatidylserine, phosphatidic acid, lysophospholipids, sphingomyelin and cardiolipin in any form, including salted or desalted, hydrogenated or partially hydrogenated, natural, semisynthetic or synthetic;
b) a co-solubilizer selected from the group consisting of charged phospholipids;
c) an isotonicity agent and;
wherein the active ingredient to phospholipid molar ratio is between 1:6.6 to 1:1 and the formulation has an average D₍ᵥₒₗ₎ particle size ranging between 2.5-30 nm for use as a medicament.

10. The pharmaceutical composition of claim 9, wherein the active ingredient is gemcitabine-5′-elaidic acid ester.

11. The pharmaceutical composition of claim 9, wherein the active ingredient to phospholipid molar ratio is between 1:6.6 to 1:2.

12. A pharmaceutical composition comprising a gemcitabine derivative of formula I: 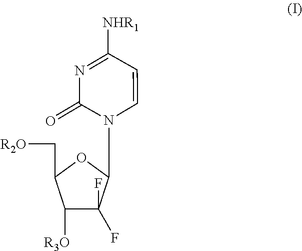
wherein R₁ and R₃ are hydrogen and R₂ is a C₁₈- or C₂₀-saturated and monounsaturated acyl group, or a pharmaceutical acceptable salt thereof as the active ingredient;
wherein the active ingredient is prepared into a formulation comprising:
a) a phospholipid solubilizer selected from the group consisting of phosphatidylcholine, phosphatidylglycerol, phosphatidylethanolamine, phosphatidylinositol, phosphatidylserine, phosphatidic acid, lysophospholipids, sphingomyelin and cardiolipin in any form, including salted or desalted, hydrogenated or partially hydrogenated, natural, semisynthetic or synthetic;
b) a co-solubilizer selected from the group consisting of charged phospholipids; and
c) an isotonicity agent;
wherein the active ingredient to phospholipid molar ratio is between 1:6.6 to 1:1 and the formulation has an average D₍ᵥₒₗ₎ particle size ranging between 2.5-30 nm, for use in treatment of cancer.

13. The pharmaceutical composition according to claim 12, wherein the active ingredient is gemcitabine-5′-elaidic ester.

14. The pharmaceutical composition according to claim 12, wherein the active ingredient to phospholipid molar ratio is between 1:6.6 to 1:2.

15. The pharmaceutical composition according to claim 12, wherein the cancer is selected from the group consisting of metastatic pancreatic cancer, non-metastatic pancreatic cancer, metastatic breast cancer, non-metastatic breast cancer, non-small cell lung cancer, uterine cancer, ovarian cancer, cervical cancer, prostate cancer, biliary tract cancer, head and neck cancer, lymphomas, myelomas, and soft tissue sarcomas.

16. The pharmaceutical composition according to claim 12, which further comprises a combination with other approved or experimental cancer therapies.

17. A method of preparation of the pharmaceutical composition according to claim 1, comprising the steps of:
a) dissolving the phospholipids and the gemcitabine derivative of formula (I) in a suitable water-miscible organic solvent;
b) injecting the organic solution obtained in step a) into an aqueous solution whereupon the lipid nanoparticles are formed;
c) subjecting the intermediate bulk solution obtained in step b) to homogenization and removal of the organic solvent.

18. The method according to claim 17, wherein the water-miscible organic solvent is selected from the group of ethanol, acetone, acetonitrile, dimethylformamide, ethylene glycol, glycerol, methanol, 1-propanol, 2-propanol or DMSO.

19. The method according to claim 17, wherein the water-miscible solvent of step a) is ethanol.

20. The pharmaceutical composition according to claim 15, which further comprises a combination with other approved or experimental cancer therapies.

21. The pharmaceutical composition according to claim 1, wherein the active ingredient to phospholipid molar ratio is 1:5 to 1:2.

22. The pharmaceutical composition of claim 9, wherein the active ingredient to phospholipid molar ratio is 1:5 to 1:2.

23. The pharmaceutical composition of claim 12, wherein the active ingredient to phospholipid molar ratio is 1:5 to 1:2.
